(19) United States
(12) Patent Application Publication
(10) Pub. No.: US 2005/0008595 A1
(43) Pub. Date: Jan. 13, 2005

(54) Cosmetic compositions with optical variability

(76) Inventors: Franck Girier Duffournier, Paris (FR); Gaetan Chevalier, Orleans (FR)

Correspondence Address: Thomas L. Irving;FINNEGAN, HENDERSON, FARABOW, GARRETT & DUNNER, L.L.P., 1300 I Street, N.W., Washington, DC, 20005-3315, US

(21) Appl. No.: 10/845,502

(22) Filed: May 14, 2004

Related U.S. Application Data
(60) Provisional application No. 60/486,911, filed on Jul. 15, 2003.

(30) Foreign Application Priority Data
May 16, 2003 (FR) 03 05932

(51) Int. Cl. A61K 7/021

(52) U.S. Cl. 424/63

(57) ABSTRACT

A composition having, for an incident illumination of 45°, a variation Dₕ of hue angle h of at least 50°, and, for example, of at least 70°, when the angle of observation is varied from −10° to +30°, and a difference (Δa*²+Δb*²)^{1/2} of at least 5 when the angle of observation is varied from −30° to −70°.

This application claims benefit of French Application No. 03 05932, filed on May 16, 2003, and U.S. Provisional Application No. 60/486,911, filed on Jul. 15, 2003.

Disclosed herein is a composition for making up a keratin material, for example the face, body, lips, nails, and various types of hair, such as eyelashes and eyebrows.

Numerous cosmetic compositions exist on the market that create variations in color by changing the angle of observation. For example, it is known practice to use diffracting pigments in nail varnish compositions to produce a rainbow effect. These pigments are sold, for example, by the company Flex Products under the name SPECTRAFLAIR. Along with the diffracting pigments, Flex Products sells interference pigments under the name CHROMAFLAIR. These interference pigments may have a structure Cr/MgF₂/Al/MgF₂/Cr.

A need still exists to create compositions with further novel optical effects, for example, novel color effects produced by varying the angle of observation.

For example, a need still exists for cosmetic compositions that have the ability to change color by varying the angle of observation over a broad range of values, for example, in order to generate a visual effect suitable for catching the eye under numerous lighting and observation conditions.

Thus, one embodiment disclosed herein relates to a composition, for example, a cosmetic composition, having increased optical variability. This composition, for an incident of illumination of 45°, has a variation Dₕ of hue angle h of at least 50°, and, for example, of at least 70°, when the angle of observation is varied from −10° to +30°, and a difference (Δa*²+Δb*²)^{1/2} of at least 5 when the angle of observation is varied from −70° to −30°.

For example, the variation Dₕ of hue angle h, is greater than or equal to 15°, and, further, for example, greater than or equal to 20°, when the angle of observation is varied from −70° to −30°.

In one embodiment, the composition disclosed herein may comprise at least one diffracting pigment. In yet another embodiment, the composition may comprise at least one goniochromatic coloring agent.

In one embodiment, the characteristic of variation in the color of the composition for angles of observation ranging from −70° to −30° can be combined with the characteristic of variation Dₕ of the hue angle h for angles of observation ranging from −10° to +30°. Combining these two characteristics results in a composition which may be capable of providing increased color variability, and thus is suitable for enabling an observer to perceive a change in color, or indeed a movement of color, under numerous conditions of observation and illumination.

Another embodiment disclosed herein relates to a composition comprising, in a physiologically acceptable medium, at least one diffracting pigment and at least one goniochromatic coloring agent.

As used herein, the expression “physiological acceptable medium” means a medium that is non-toxic and suitable for being applied to a human keratin material, such as skin, lips, nails, and hair.

As used herein, the term “diffracting pigment” means pigments that can produce variations in color depending on the angle of observation, when it is illuminated in white light. The variation of color is caused by the presence of a structure that diffracts light.

As used herein, the term “goniochromatic coloring agent” means an agent that can produce variations in color, depending on the angle of observation, by means other than diffraction grating alone.

As used herein, the term “cosmetic composition” means a composition as defined in the Jun. 14, 1993 EEC Council Directive 93/35.

Compositions comprising both at least one diffracting pigment and at least one goniochromatic coloring agent have been proposed in the article “Pigments Exhibiting a Combination of Thin Film and Diffractive Light Interference” by Alberto Argoitia, in the AIMCAL 2002 Fall Technical Conference Meeting, Sedona, Ariz., Oct. 20-23, 2002. The at least one goniochromatic coloring agent used in the compositions disclosed therein are pigments that change from green to blue and comprise a deposit of aluminium, 80 nanometers (nm) thick.

However, these compositions may not result in a completely satisfactory optical effect. Thus, a need still exists for a composition, such as a cosmetic composition, comprising, in a physiologically acceptable medium, at least one diffracting pigment and at least one goniochromatic coloring agent, wherein the composition is capable of providing increased attractiveness, for example, the composition is capable of producing different colors over a wide range of values for the angle of observation.

Another embodiment disclosed herein relates to a composition comprising the at least one goniochromatic coloring agent, wherein the at least one goniochromatic coloring agent is sufficiently transparent for it to be possible, under incident illumination at +45°, to observe a variation Dₕ of hue angle h of at least 50°, for example, of at least 70°, when the angle of observation is varied from −10° to +30°, and a difference (Δa*²+Δb*²)^{1/2} of at least 5 when the angle of observation is varied from −70° to −30°.

In the above embodiment, the variation Dₕ in hue angle h between the angles of observation ranging from −10° to +30° may be induced by the at least one diffracting pigment. The at least one diffracting pigment can generate a rainbow effect. Whereas the variation in color that is observed between the angles of observation ranging from −30° to −70° may come from the at least one goniochromatic coloring agent. Because the at least one goniochromatic coloring agent is sufficiently transparent, the rainbow effect can continue, in one embodiment, to be observed even though the at least one diffracting pigment is dispersed within the composition that comprises the at least one goniochromatic coloring agent, even when the at least one goniochromatic coloring agent is present in a non-negligible quantity.

In one embodiment, the at least one goniochromatic coloring agent does not comprise a reflecting metal deposit, for example, aluminum, but has a structure comprising oxides, thus making it possible to obtain a pigment of reduced opaqueness.

For example, in one embodiment, the at least one goniochromatic coloring agent is not chosen from pigments having a dimension greater than 80 microns (μm), thus making it possible to further reduce the risk of the at least one goniochromatic coloring agent impeding reception of the changing color induced by the at least one diffracting pigment.

The at least one goniochromatic coloring agent may thus, in one embodiment, be chosen from pigments having a dimension that is less than or equal to 80 μm, for example, less than or equal to 70 μm, and, further, for example, less than or equal to 50 μm, while nevertheless being greater than or equal to 4 μm, and greater than or equal to 6 μm, for example.

The weight ratio of the at least one diffracting pigment to the at least one goniochromatic coloring agent may range from 15/85 to 85/15, further, for example, from 20/80 to 80/20, even further, for example, from 40/60 to 60/40, and may, for example, be 50/50. These ratios may be favorable for producing both a marked rainbow and goniochromatic effect.

The composition disclosed herein may be provided in various forms. For example, the composition may be providing in forms chosen from nail varnish compositions; makeup compositions for the lips, such as lipsticks and lip glosses; mascaras; and foundations.

Another embodiment disclosed herein relates to the use of at least one non-opaque goniochromatic coloring agent and at least one diffracting pigment for making up at least one keratin material chosen from skin, lips, nails, and hair.

Yet another embodiment disclosed herein relates to a method of making up at least one keratin material chosen from skin, lips, nails, and hair, comprising simultaneous applying to the at least one keratin material at least one non-opaque goniochromatic coloring agent and at least one diffracting pigment.

Another disclosed embodiment relates to a composition, for example, a nail varnish composition, comprising, in a physiologically acceptable medium, from 0.5% to 2.5% by weight, for example, 1% by weight, relative to the total weight of the composition, of at least one diffracting pigment having a structure MgF₂/Al/MgF₂ and from 0.5% to 2.5% by weight, for example, 1% by weight, relative to the total weight of the composition, of at least one goniochromatic coloring agent having a structure Fe₂O₃/SiO₂/Fe₂O₃/SiO₂/Fe₂O₃, wherein the weight ratio of the at least one diffracting pigment to the at least one goniochromatic coloring agent ranges from 15/85 to 85/15, for example, from 40/60 to 60/40, and, even further, for example, may be 50/50.

Another embodiment disclosed herein relates to a composition comprising, in a physiologically acceptable medium, from 0.5% to 2.5% by weight, for example, 1% by weight, relative to the total weight of the composition, of at least one diffracting pigment having a structure MgF₂/Al/MgF₂ and from 0.5% to 2.5% by weight, for example, from 0.75% to 2% by weight, and, for example,1% by weight, relative to the total weight of the composition, of the at least one goniochromatic coloring agent having a structure Fe₂O₃/SiO₂/mica-oxide/SiO₂/Fe₂O₃, wherein the weight ratio of the at least one diffracting pigment to the at least one goniochromatic coloring agent ranges from 15/85 to 85/15, for example, from 40/60 to 60/40 and, may be, for example, 50/50.

Yet another embodiment disclosed herein relates to a composition comprising, in a physiologically acceptable medium, from 0.5% to 2.5% by weight, for example, 1% by weight, relative to the total weight of the composition, of at least one diffracting pigment having a structure MgF₂/Al/MgF₂ and 0.5% to 2.5% by weight, for example,1% by weight, relative to the total weight of the composition, of at least one goniochromatic coloring agent having a structure TiO₂/SiO₂/TiO₂, wherein the weight ratio of the at least one diffracting pigment to the at least one goniochromatic coloring agent ranges from 15/85 to 85/15, for example, from 40/60 to 60/40, and, for example, may be 50/50.

Another embodiment disclosed herein relates to a composition comprising, in a physiologically acceptable medium, from 0.5% to 2.5% by weight, for example,1% by weight, relative to the total weight of the composition, of at least one diffracting pigment having a structure MgF₂/Al/MgF₂ and 0.5% to 2.5% by weight, for example, 1% by weight, relative to the total weight of the composition, of the at least one goniochromatic coloring agent having a structure TiO₂/Al₂O₃/TiO₂, wherein the weight ratio of the at least one diffracting pigment to the at least one goniochromatic coloring agent ranges from 15/85 to 85/15, from 40/60 to 60/40, and may be, for example, 50/50.

Measuring Variation Dₕ of Hue Angle h and of the Difference (Δa*²+Δb*2)^{1/2}

The measurement of variation Dₕ of hue angle h and the difference (Δa*²+Δb*2)^{1/2} is performed after the composition has been spread to a thickness of 150 μm, using an automatic spreader, onto the black background of a conventional contrast card, for example, an Erichsen card of reference Typ 24/5.

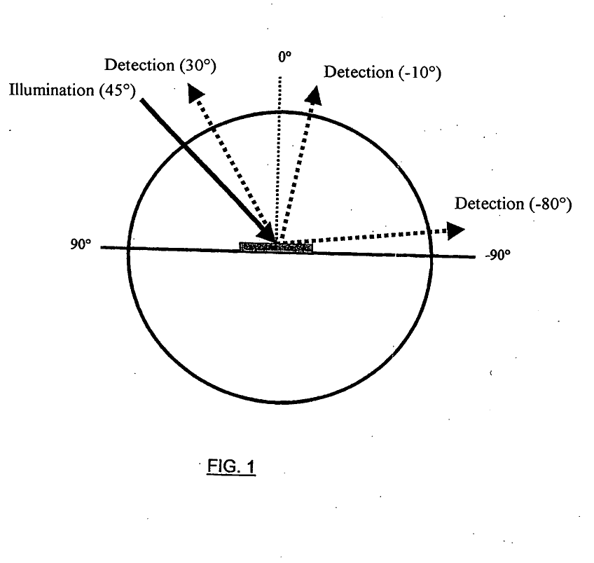

The measurement is performed with a spectrogonioreflectometer, for example, an Instrument Systems spectrogonioreflectometer of reference GON 360 Goniometer, with incident lighting at 45° and with illuminant D₆₅, using the scheme shown in FIG. 1. The apparatus is in “10° observer” mode, with the analyzed spectrum ranging from 400 nm to 700 nm (at a pitch of 5 nm).

A negative angle of observation corresponds in this case to observation in the half-plane opposite to the half-plane from which the light comes, normal to the illuminated surface, as can be seen in FIG. 1.

Unless stated otherwise, it is assumed throughout the description that measurements are performed under the conditions of spreading to a thickness of 150 μm, and after the composition has dried.

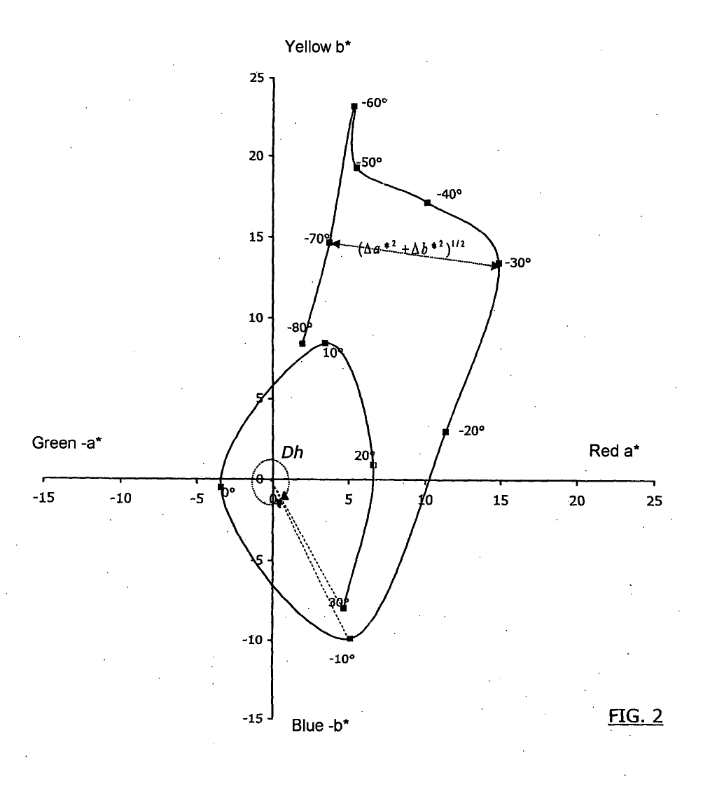

FIG. 2 shows the color path of the composition disclosed herein plotted on a 1976 CIE L*a*b* plane, as obtained with the spectrogonioreflectometer mentioned above.

In FIG. 2, the variation Dₕ of hue angle h is almost 360° between observation angles having values ranging from −10° to 30°. The variation Dₕ corresponds to the angular sector swept by a radius starting from the achromatic point O and having its end describing the color path, with the scale being the same for the −a*a* and the −b*b* axes. A variation in Dₕ of at least 360° means that the path passes through all four quadrants of the a*b* plane.

The difference ΔE_{ab}* between the points corresponding to the angles −30° and −70° is defined by the equation:

$$\begin{matrix}Δ {E}_{ab}^{★} = \sqrt{{( {a}_{- 70 °}^{★} - {a}_{- 30 °}^{★} )}^{2} + {( {b}_{- 70 °}^{★} - {b}_{- 30 °}^{★} )}^{2}} \\ = {( Δ {a}^{★2} + Δ {b}^{★2} )}^{1 / 2}\end{matrix}$$

In the example described, the following values apply:
a*_{-70°}=3.74
a*_{-30°}=14.92
b*_{-70°}=14.68
b*_{-30°}=13.44
giving
ΔE_{ab}*=11.25
and Dₕ≈360°

One of the points corresponding to the angles ranging from −30° to −70°, or indeed both of them, may lie in one of the quadrants corresponding to a*>0, as can be seen in FIGS. 2 to 5.

### EXAMPLES OF DIFFRACTING PIGMENTS

The composition disclosed herein comprises at least one diffracting pigment. It is possible to use a single type of diffracting pigment for ease of preparation.

The at least one diffracting pigment may comprise a diffraction grating that is capable, for example, of diffracting an incident wave of monochromatic light in defined directions.

The diffraction grating may comprise a periodic pattern, for example, a line, with the distance between two adjacent patterns being of the same order of magnitude as the wavelength of the incident light.

When the incident light is polychromatic, the diffraction grating separates the various components of the light spectrum and produces a rainbow effect.

Concerning the structure of at least one diffracting pigment, reference can be made to the article entitled “Pigments Exhibiting Diffractive Effects” by Alberto Argoitia and Matt Witzman, 2002, Society of Vacuum Coaters, 45th Annual Technical: Conference Proceedings, 2002, the disclosure which relates to the structure of the at least one diffracting pigment is incorporated herein by reference.

The at least one diffracting pigment may be made with patterns having different profiles, for example, profiles chosen from triangular, optionally symmetrical, crenellated, of optionally constant width, and sinusoidal profiles.

The spatial frequency of the grating and the depth of the patterns should be chosen as a function of the degree of separation desired for different orders. For example, the spatial frequency may range from 500 lines per mm to 3000 lines per mm.

In some embodiments, each particle of the at least one diffracting pigment may be flat in shape, and, for example, may be in the form of a platelet.

In other embodiments, a single particle of the at least one diffracting pigment may have two crossed diffraction gratings, that may optionally be perpendicular.

In another embodiment, the at least one diffracting pigment may comprise at least one layer of at least one reflecting material covered on at least one side by at least one layer of at least one dielectric material. The at least one dielectric material may impart better stiffness and durability on the at least one diffracting pigment. The at least one dielectric material may, for example, be chosen from MgF₂; SiO₂; Al₂O₃; AlF₃; CeF₃; LaF₃; NdF₃; SmF2; BaF₂; CaF₂; and LiF, as used herein, “at least one” would include mixtures and associations. The at least one reflecting material may be chosen, for example, from metals, metal alloys, and non-metallic reflecting materials. Examples of a suitable at least one reflecting material include Al; Ag; Cu; Au; Pt; Sn; Ti; Pd; Ni; Co; Rd; Nb; Cr; and compounds, associations, and alloys thereof. The at least one reflecting material may, alone, make up the at least one diffracting pigment, in which case the at least one diffracting pigment comprises a single layer.

In some embodiments, the at least one diffracting pigment is a multilayer structure pigment having a core comprising at least one dielectric material covered in at least one layer of at least one reflecting material on at least one side, and possibly completely encapsulating the core. The at least one dielectric material may also cover the at least one layer of the at least one reflecting material. The at least one dielectric material used may, for example, be inorganic, and may be chosen, for example, from metal fluorides; metal oxides; metal sulfides; metal nitrides; and metal carbides. The at least one dielectric material may be crystalline; semicrystalline, or amorphous. In this configuration, the at least one dielectric material may be chosen, for example, from MgF₂; SiO; SiO₂; Al₂O₃; TiO₂; WO; AlN; BN; B₄C; WC; TiC; TiN; N₄Si₃; ZnS; particles of glass; and particles of diamond-type carbons.

In one embodiment, the at least one diffracting pigment comprising the at least one dielectric material may be chosen from preformed dielectric and ceramic materials such as a naturally lamellar mineral, for example, peroskovite mica and talc, and synthetic lamellae made of the following: glass; alumina; SiO₂; carbon; iron/mica oxide; mica covered in BN; BC; graphite; and bismuth oxychloride.

Instead of the at least one layer of the at least one dielectric material, other materials that improve mechanical properties may be suitable.. Examples of such materials include at least one of silicone; metal silicides; semiconductive compounds made with elements from groups II, IV, and V: metals having a body-centered cubic crystal structure; cermet compounds and compositions; and semiconductive glasses.

The at least one diffracting pigment used may be chosen from those described in U.S. patent application No. U.S. 2003/0031870 published on Feb. 13, 2003, the disclosure relating to the at least one diffracting pigment is incorporated herein by reference.

For example, the at least one diffracting pigment may have a structure MgF₂/Al/MgF₂. The at least one diffracting pigment having this structure is sold by Flex Products under the name SPECTRAFLAIR 1400 Pigment Silver and SPECTRAFLAIR 1400 Pigment Silver FG. The MgF₂ may be present in an amount ranging from 80% to 95% by weight, relative to the total weight of the at least one diffracting pigment.

The at least one diffracting pigment may be present in an amount equal to at least 5% by weight, relative to the total weight of the composition. Further, for example, the at least one diffracting pigment may be present in an amount ranging, for example, from 0.1% to 5%, by weight, further, for example, from 0.5% to 5% by weight, further, for example, from 0.5% to 2.5% by weight, and may, for example, be 1% by weight, relative to the total weight of the composition, when it is, for example, to be applied to the nails.

The dimension of the at least one diffracting pigment may range from 5 μm to 200 μm, for example, from 5 μm to 100 μm, and, even further, for example, from 5 μm to 30 μm.

As used herein, the term “dimension” means the statistical grain size distribution for half of the population, known as D₅₀.

The thickness of the at least one diffracting pigment particles may be less than or equal to 3 μm, for example, less than or equal to 2 μm, and may be 1 μm.

In one embodiment, the at least one diffracting pigment should be chosen in such a manner as to make it possible, under incident illumination at 45° and for an angle of observation ranging from −10° to +30°, to observe the above-specified variation Dₕ in the hue angle h of the composition spread to a thickness of 150 μm, of at least 50°, for example, of at least 70°, even further, for example, of at least 80° or 90°, and further, for example, of at least 100° on the 1976 CIE L*a*b* plane. For example, for a nail varnish the variation Dₕ may be at least 180°, further, for example, at least 270°, and, further, for example, at least 360°.

For gloss, good results have been obtained, for example, with Dₕ varying over at least 90° between the angles of observation ranging from −10° to 30°.

### EXAMPLES OF GONIOCHROMATIC COLORING AGENTS

The at least one goniochromatic coloring agent may, for example, be chosen from interfering multilayer structure pigments.

Examples of goniochromatic coloring agents are disclosed in EP 1 195 155 and EP 1 249 226, the disclosure is incorporated herein by reference.

When the at least one goniochromatic coloring agent is present at 1% by weight in, for example, a nail varnish base and spread to a thickness of 150 μm, it enables a variation of Dₕ of hue angle h to be obtained that is greater than or equal to 30°, for example, at least equal to 60°, when the angle of observation is varied from −80° to 0°.

The composition disclosed herein may comprise a plurality of the at least one goniochromatic coloring agent, but it is possible to use a single goniochromatic coloring agent in order to facilitate preparation.

Depending on the nature of the composition, the at least one goniochromatic coloring agent may be present in an amount ranging, for example, from 0.5% to 60% by weight, for example, from 0.5% to 2.5% by weight, relative to the total weight of the composition. In one embodiment, the at least one goniochromatic coloring agent may be present in amount equal to less than 5% by weight, relative to the total weight of the composition. For example, a nail varnish composition may comprise from 0.1% to 5% by weight, of the at least one goniochromatic coloring agent; a foundation makeup may comprise from 1% to 15% by weight, for example, and, further, for example, a lipstick may comprise from 1% to 10% by weight, of the at least one goniochromatic coloring agent relative to the total weight of the composition.

With nail varnishes, very satisfactory results have been obtained with a composition comprising 1% by weight of the at least one goniochromatic coloring agent and 1% by weight of the at least one diffracting pigment, the percentages being measured relative to the total weight of the composition.

The at least one goniochromatic coloring agent may, for example, have a dimension of less than 80 μm, for example, ranging from 4 μm to 80 μm, and, further, for example, ranging from 6 μm to 70 μm.

In one embodiment disclosed herein the at least one goniochromatic coloring agent is not opaque, i.e. it is sufficiently transparent for it to be possible with illumination of 45° and with the angle of observation varying from −10° to +30° to observe a rainbow effect representative of a sufficient variation Dₕ in the hue angle h of the composition disclosed herein in the 1976 CIE L*a*b* plane. For example, for a nail varnish, the variation may be at least 120°, further, for example, at least 180°, further, for example, at least 270°, and, further, for example, may be 360°, this variation corresponding to the rainbow effect induced by the at least one diffracting pigment. For gloss, this varination may, for example, be at least 90°.

The at least one goniochromatic coloring agent also makes it possible to obtain a difference ΔE_{ab}*=(Δa*²+Δb*²)^{1/2} for the composition as measured in the L*a*b* plane of the 1976 CIE color space of at least 5, further, for example, at least 8, for example, of at least 10, for example, of at least 15, and, for example, of at least 20, with this difference being measured as described above.

For example, the at least one goniochromatic coloring agent may be chosen from pigments having non-opaque interfering multilayer structures and non-opaque liquid crystal coloring agents.

When the at least one gonichromatic coloring agent is a multilayer interfering structure pigment, it may comprise, for example, at least two layers, wherein each layer comprises at least one material chosen from: MgF₂; CeF₃; ZnS; ZnSe; Si; SiO₂; Ge; Te; Fe₂O₃; Pt; Va; Al₂O₃; MgO; Y₂O₃; S₂O₃; SiO; HfO₂; ZrO₂; CeO₂; Ng₂O₃; Ta₂O₅; TiO₂; Ag; Al; Au; Cu; Rb; Ti; Ta; W; Zn; MoS₂; cryolite; alloys, and polymers, providing that they are not too opaque.

Each layer may be non-metallic, in order to avoid spoiling the transparency of the structure.

Relative to a central layer, each multilayer structure may optionally be symmetrical, in terms of the chemical nature of the stacked layers. Depending on the thicknesses and the chemical nature of the various layers, different effects may be obtained.

Examples of symmetrical interference multilayer structures suitable for use in the compositions disclosed herein include: Fe₂O₃/SiO₂/Fe₂O₃/SiO₂/Fe₂O₃, a pigment having this structure is sold by BASF under the name SICOPEARL; MoS₂/SiO₂/mica-oxide/SiO₂/MoS₂; Fe₂O₃/SiO₂/mica-oxide/SiO₂/Fe₂O₃; TiO₂/SiO₂/TiO₂; TiO₂/Al₂O₃/TiO₂; pigments with these structures are sold by Merck (Darmstadt) under the name XIRONA.

For example, the at least one gionochromatic coloring agent may be chosen from liquid crystal coloring agents comprising silicones and cellulose ethers having mesomorphic groups grafted thereto.

Examples of liquid crystal goniochromatic particles include those sold by Chenix and Wacker under the name HELICONE HC.

The at least one goniochromatic coloring agent may also be chosen from certain lusters, pigments producing effects on a synthetic substrate, for example, a substrate of at least one of the following types: alumina; silica; borosilicate; iron oxide; aluminum; and interfering holographic flakes coming from a film of polyterephthalate.

The composition may also comprise dispersed goniochromatic fibers. Such fibers may, for example, have a length shorter than 80 μm.

Other Coloring Agents

The composition disclosed herein may also comprise at least one additional coloring agent, for example, chosen from dyes, such as liposoluble and hydrosoluble dyes; monochromatic pigments; and certain luster pigments conventionally used in cosmetic compositions.

Examples of luster pigments include natural mica covered in titanium oxide, iron oxide, natural pigment, and bismuth oxychloride. Amongst the luster pigments available on the market, examples include pigments sold under the name TIMICA and FLAMENCO by Engelhard and TIMIRON sold by Merck.

The at least one additional coloring agent may also be chosen from non-goniochromatic and non-diffracting coloring agents. The non-goniochromatic and non-diffracting coloring agents may be chosen, for example, from dyes and monochromatic pigments, and can serve, for example, to correct the hues produced by the above-described at least-one diffracting pigment and the at least one goniochromatic coloring agent, so as to prevent the appearance of a color that is deemed to be non-desirable. The non-goniochromatic and non-diffracting coloring agents may also be present in the composition in order to give it a desired color under certain conditions of observation.

The at least one additional coloring agent other than the at least one diffracting pigment may be chosen in such a manner that the color path in the 1976 CIE a*b* plane passes through all four quadrants and “surrounds” the achromatic point, as can be seen in FIG. 2, for example, when the angle of observation varies from −10° to 30°.

Physiologically Acceptable Medium

The physiologically acceptable medium should be adapted to the nature of the medium onto which the composition is to be applied, and also to the form in which the composition is to be packaged, for example, depending on whether it is solid or fluid at ambient treatment and atmospheric pressure.

The composition disclosed herein may comprise at least one medium chosen from aqueous and fatty cosmetic mediums.

The composition may comprise water or mixtures of water and at least one hydrophilic organic solvent chosen, for example, from alcohols, such as linear and branched lower monoalcohols comprising from 2 to 5 carbon atoms such as ethanol, isopropanol, and n-propanol and polyols, such as glycerine, diglycerine, glycol propylene, sorbitoi, glycol penthylene, and glycol polyethylenes. The at least one hydrophilic organic solvent may also comprise C₂ ethers and hydrophilic C₂-C₄ aldehydes. The water or the mixtures of water and the at least one hydrophilic organic solvent may be present in the composition disclosed herein in an amount ranging from 0% to 90% by weight, for example, from 0.1% to 90% by weight, further, for example, from 0% to 60% by weight, and, even further, for example, from 0.1% to 60% by weight, relative to the total weight of the composition.

The composition may also comprise at least one fatty phase, for example, comprising at least one fatty substance chosen from fats that are liquid at ambient temperature (in general 25° C.°) and fats that are solid at ambient temperature, such as semisolid fats and gums. The at least one fatty phase may also comprise at least one lipophilic organic solvent.

Examples of fats that are liquid at ambient temperature, often known as oils, that can be used in the composition disclosed herein include vegetable hydrocarbon oils such as liquid triglycerides of fatty acids comprising from 4 to 10 carbon atoms such as triglycerides of heptanoate and octanoate acids; sunflower, corn (maize), soy bean, grape pip, sesame, apricot, macademia, castor-oil beans, and avocado oils; triglycerides of capyrlic/capric acids; jojoba oil; karite butter; linear and branched hydrocarbons of mineral and synthetic origin such as paraffin oils and derivatives thereof; Vaseline; polydecenes; hydrogenated polyisobutene such as parleam; synthetic esters and ethers, for example, of fatty acids, such as Purcellin oil, isopropyl myristate, ethyl-2-hexyl palmitate, octyl-2-dodecyle stearate, octyl-2-dodecyl erucate, and isostearyl stearate; hydroxyl esters such as isotearyl lactate, octylhydroxystearate, octododecyl hydroxystearate, diisotearylmalate, triisocetyl citrate, heptanoates, octanoates, and fatty acid decanoates; polyol esters such as propylene glycol dioctanoate, neopentylglycol diheptanoate, and diethyleneglycol diisononanoate; esters of pentaerythritol; fatty acids comprising from 12 to 26 carbon atoms, such as octyldodecanol, 2-butyloctanol, 2-hexyldecanol, 2-undecylpentadecanol, and oleic alcohol; partially hydrocarbonated and/or siliconized fluorinated oils; and silicone oils such as polymethylsiloxanes (PDMS) which may be volatile or otherwise, linear and cyclic, liquid and semisolid at ambient temperature, such as cyclomethicones, dimethicones, optionally comprising at least one phenyl group, such as phenyl trimethicones, phenyltrimethylsiloxydiphenyl siloxanes, diphenylmethyldimethyl-trisiloxanes, diphenyl dimethicones, phenyl dimethicones, and polymethylphenyl siloxanes.

These oils may be present in an amount ranging from 0.01% to 90% by weight, and, for example, from 0.1% to 85% by weight, relative to the total weight of the composition.

The composition disclosed herein may also comprise at least one physiologically acceptable organic solvent. The at least one physiologically acceptable solvent may be present in an amount ranging from 0 to 90% by weight, for example, from 0 to 60% by weight, and, even further, for example, from 0.1% to 30% by weight, relative to the total weight of the composition.

The presence of the at least one physiologically acceptable organic solvent may, for example, be suitable for making up the nails. The composition may then be in the form of a nail varnish. The at least one physiologically acceptable organic solvent may be present in the composition in an amount ranging from 30% to 99% by weight, relative to the total weight of the composition, and, for example, from 60% to 90% by weight, relative to the total weight of the composition.

When the physiologically acceptable medium of the composition comprises at least one liquid phase it may, for example, be an organic liquid phase wherein the water is dispersed or emulsioned.

The composition disclosed herein may comprise at least one continuous fatty phase, possibly comprising no more than 5% by weight, of water, for example, no more than 1% by weight of water, relative to its total weight of the composition. The composition may, for example, be in anhydrous form.

Fillers

The composition may also comprise at least one filler.

As used herein, the term “fillers” means particles of any kind that are insoluble in the medium of the composition, regardless of the temperature at which the composition is prepared. Such fillers may serve, for example, to modify the rheology or the texture of the composition.

The at least one filler may, for example, be chosen from talc, mica, silica, kaolin, and polyamide powders, such as NYLON and ORGASOL from Atochem.

In one embodiment, the at least one filler and the at least one additional coloring agent should not harm the looked-for optical effects.

Cosmetic Active Agents

The composition may also comprise at least one active agent chosen from cosmetic, dermatological, hygienic, and pharmaceutical active agents.

The at least one active agent may, for example, be chosen from moisturizers (polyol such as glycerine); vitamins (C, A, E, F, B, and PP); essential fatty acids; essential oils; ceramides; spingolipids; sun filters that are liposoluble and in the form of nano-particles; and active agents specific for treating the skin, such as protective agents, antibacterial agents, antiwrinkle agents, etc. The at least one active agent may be present in an amount ranging from 0 to 20% by weight, and, for example, from 0.001% to 15% by weight, relative to the total weight of the composition.

Other Ingredients

The composition may also comprise at least one adjuvant that is commonly used in cosmetics. The at least one adjuvant may, for example, be chosen from thickeners, wetting agents, oligo-elements, moisturizers, softeners, sequestering agents, fragrances, alkaline and acid agents, preservatives, antioxidants, and UV filters.

In addition, depending on the type of application intended, the composition may further comprise other ingredients that are conventionally used in the fields in question, which are present in quantities appropriate for the desired form.

Physical Form

The composition may be provided in any form that is normally used for topical application. For example, the composition may be provided in a form chosen from anhydrous forms; oily and aqueous solutions; oily and aqueous gels; oil-in-water and water-in-oil emulsions; multiple emulsions; and dispersions of oil in water by means of vesicles situated at the oil/water interface.

The composition disclosed herein may be in a form chosen from powders; liquids, solids and semisouids, for example, in the form of a product that has been cast into a stick, a cup, paste, cream of greater or smaller fluidity, and a film deposited on a flexible sheet.

The composition disclosed herein may also be provided in a form chosen from nail varnishes; lipsticks; liquid glosses; red pastes for the lips; rouge; lip pencils, solid and fluid foundation makeup compositions; preparations against bags under the eyes and around the eyes; eyeliners; mascaras; eye shadows; makeup compositions for the body and the hair; and sun shield and skin-coloring preparations.

The composition disclosed herein may be obtained using the methods of preparation that are conventionally used in cosmetics.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, the numerical values set forth in the specific examples are reported as precisely as possible. Any numerical value, however, inherently contain certain errors necessarily resulting from the standard deviation found in their respective testing measurements. The following examples are intended to illustrate the present disclosure without limiting the scope as a result.

### EXAMPLE 1

Nail Varnish

A nail varnish was made having the following formulation (proportions by weight):

| | |
|---|---|
| diffracting pigment* | 1% |
| goniochromatic coloring agent** | 1% |
| nail varnish base*** | qsp 100% |

*Spectraflair 1400 Silver (Flex Products)
**Sicopearl (BASF)
***The nail varnish base had the following ingredients (%):

| | |
|---|---|
| nitrocellulose | 10 |
| plasticizers and resins | 15 |
| rheological agent | 1.5 |
| ethyl acetate, butayl acetate | qsp 100% |

A composition was obtained having the color path shown in FIG. 2.

It can be seen that for a variation in the angle of observation lying in the ranging from −10° to 30°, a variation Dₕ is observed of the hue angle h of 360° with a difference ΔE_{ab}* of greater than 5.

With such varnish, large variability was obtained in its color over a large range of values for the angle of observation, and the presence of the at least one goniochromatic pigment affect the rainbow effect.

### EXAMPLE 2

Nail varnish

| | |
|---|---|
| Diffracting pigment* | 1% |
| Goniochromatic coloring agent** | 1% |
| Nail varnish base*** | qsp 100% |

*Spectraflair 1400 Silver (Flex Products)
**Xirona Magic Mauve (Merck)
***The nail varnish base contained the following ingredients (%):

| | |
|---|---|
| nitrocellulose | 10 |
| plasticizers and resins | 15 |
| rheological agent | 1.5 |
| ethyl acetate, butayl acetate | qsp 100% |

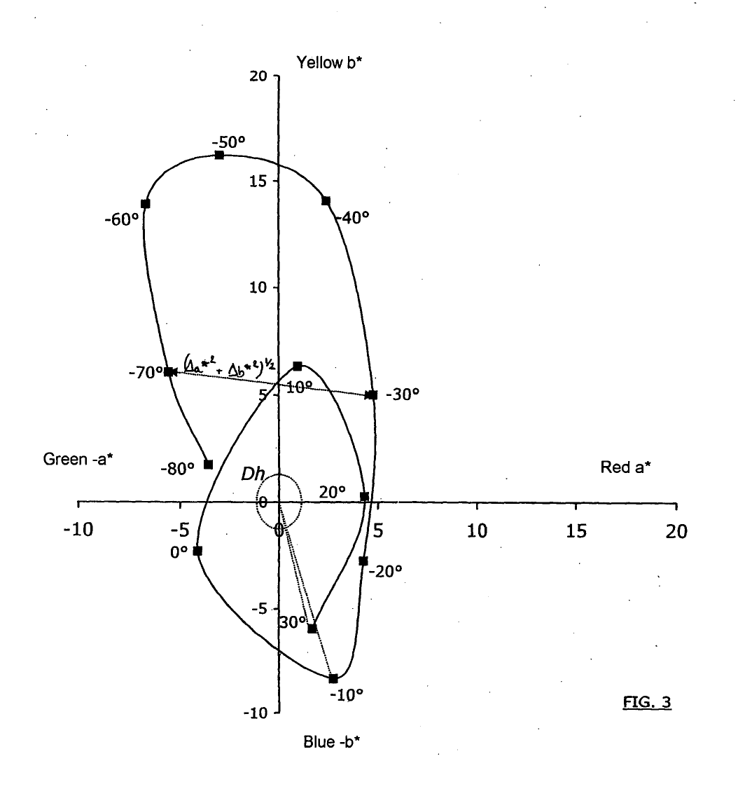

The color path observed is shown in FIG. 3, with large variation in color with a varying angle of observation.

### EXAMPLE 3

Nail varnish

| | |
|---|---|
| Diffracting pigment* | 1% |
| Goniochromatic coloring agent** | 1% |
| Nail varnish base*** | qsp 100% |

*Spectraflair 1400 Silver (Flex Products)
**Xirona Caribbean Blue (Merck)
***The nail varnish base contained the following ingredients (%):

| | |
|---|---|
| nitrocellulose | 10 |
| plasticizers and resins | 15 |
| rheological agent | 1.5 |
| ethyl acetate, butayl acetate | qsp 100% |

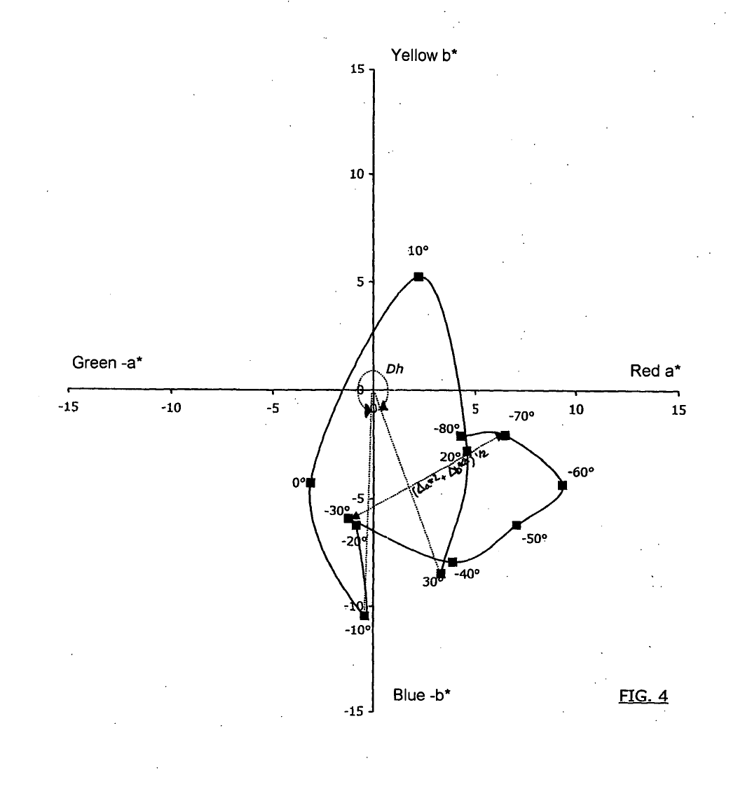

The color path of FIG. 4 was observed.

### EXAMPLE 4

Gloss

| | |
|---|---|
| Diffracting pigment* | 1% |
| Goniochromatic coloring agent** | 1% |
| Gloss base*** | qsp 100% |

*Spectraflair 1400 Silver (Flex Products)
**Sicopearl (BASF)
***The gloss base contained the following ingredients (%):

| | |
|---|---|
| bis-diglyceryl poyacryladipate-2 | 19.4 |
| diiostearyl malate | 10.5 |
| tridecyl trimellitate | 11.1 |
| C₁₈₋₃₆ acid triglyceride | 21.1 |
| silica dimethyl silylate | 8.9 |
| polybutene | 13.3 |
| pentaerythrityl tetra isostearate | 14.4 |
| fragrance, preservative | qs |

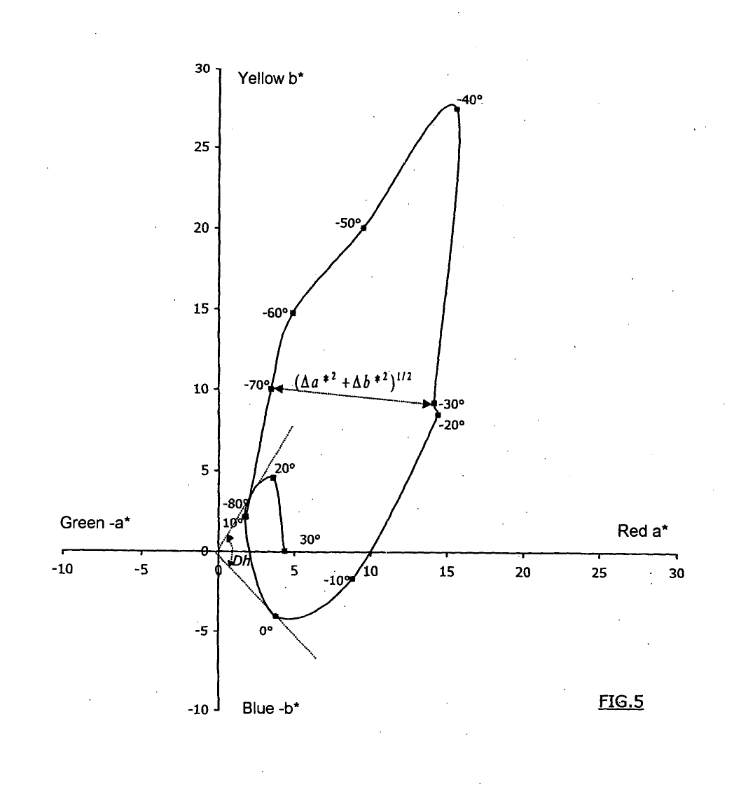

The color path of FIG. 5 was observed.

A variation Dₕ in hue angle h of 100° could be measured for angle of observation varying over the range −10 to 30°.

Naturally, the disclosed embodiments are not limited to the examples given above, and other pigments also can be used.

Throughout the description, including in the claims, the term “comprising a” should be understood as being synonymous with “comprising at least one”, unless specified to the contrary.

Although the present disclosure has been described with reference to particular embodiments, it is to be understood that these embodiments are merely illustrative of the principles and applications, without, however, being limiting in nature. It is therefore to be understood that numerous modifications may be made to the illustrative embodiments and that other arrangements may be devised without departing from the spirit and scope of the present disclosure as defined by the claims.

1. A composition having, for an incidence of illumation of 45°, a variation Dₕ of hue angle h of at least 50°, when the angle of observation is varied from −10° to +30°, and a difference (Δa*²+Δb*2)^{1/2} of at least 5 when the angle of observation is varied from −70° to −30°.

2. The composition according to claim 1, wherein the variation Dₕ of hue angle h is at least 70°.

3. The composition according to claim 1, further comprising, in a physiologically acceptable medium, at least one diffracting pigment.

4. The composition according to claim 1, further comprising, in a physiologically acceptable medium, at least one diffracting pigment and at least one goniochromatic coloring agent.

5. The composition according to claim 4, wherein the at least one goniochromatic coloring agent is sufficiently transparent to enable the variation Dₕ of hue angle h to be observed.

6. The composition according to claim 4, wherein the at least one goniochromatic coloring agent is chosen from pigments having a dimension less than or equal to 80 μm.

7. The composition according to claim 6, wherein the at least one goniochromatic coloring agent is chosen from pigments having a dimension less than or equal to 70 μm.

8. The composition according to claim 7, wherein the at least one goniochromatic coloring agent is chosen from pigments having a dimension less than or equal to 50 μm.

9. The composition according to claim 6, wherein the at least one goniochromatic coloring agent is chosen from pigments having a dimension greater than or equal to 4 μm.

10. The composition according to claim 9, wherein the at least one goniochromatic coloring agent is chosen from pigments having a dimension greater than or equal to 6 μm.

11. The composition according to claim 1, wherein the variation Dₕ of hue angle h is greater than or equal to 80° for in angle of observation varying from −10° to +30°.

12. The composition according to claim 11, wherein the variation Dₕ of hue angle h is greater than or equal to 90° for an angle of observation varying from −10° to +30°.

13. The composition according to claim 12, wherein the variation Dₕ of hue angle h is greater than or equal to 100° for an angle of observation varying from −10° to +30°.

14. The composition according to claim 13, wherein the variation Dₕ of hue angle h is greater than or equal to 180° for an angle of observation varying from −10° to +30°.

15. The composition according to claim 14, wherein the composition is provided in the form of a nail varnish.

16. The composition according to claim 14, wherein the variation Dₕ of hue angle h is greater than or equal to 270° for in angle of observation varying from −10° to +30°.

17. The composition according to claim 16, wherein the variation Dₕ of hue angle h is equal to 360° for an angle of observation varying from −10° to +30°.

18. The composition according to claim 1, wherein the difference (Δa*²+Δb*2)^{1/2} is at least 8, for an angle of observation varying from −70° to −30°.

19. The composition according to claim 18, wherein the difference (Δa*²+Δb*2)^{1/2} is at least 10, for an angle of observation varying from −70° to −30°.

20. The composition according to claim 19, wherein the difference (Δa*²+Δb*²)^{1/2} is at least 15, for an angle of observation varying from −70° to −30°.

21. The composition according to claim 20, wherein the difference (Δa*² +Δb*²)^{1/2} is at least 20, for an angle of observation varying from −70° to −30°.

22. The composition according to claim 1, wherein the variation Dₕ in hue angle h for angles of observation varying from −70° to −30° is greater than or equal to 15°.

23. The composition according to claim 22, wherein the variation Dₕ in hue angle h for angles of observation varying from −70° to −30° is greater than or equal to 20°.

24. The composition according to claim 4, wherein the weight ratio of the at least one diffracting pigment to the at least one goniochromatic coloring agent ranges from 15/85 to 85/15.

25. The composition according to claim 24, wherein the weight ratio of the at least one diffracting pigment to the at least one goniochromatic coloring agent ranges from 20/80 to 80/20.

26. The composition according to claim 25, wherein the weight ratio of the at least one diffracting pigment to the at least one goniochromatic coloring agent ranges from 40/60 to 60/40.

27. The composition according to claim 26, wherein the weight ratio of the at least one diffracting pigment to the at least one goniochromatic coloring agent is 50/50.

28. The composition according to claim 4, wherein the at least one diffracting pigment is present in an amount equal to at least 5% by weight, relative to the total weight of the composition.

29. The composition according to claim 4, wherein the at least one diffracting pigment is present in an amount ranging from 0.1% to 5% by weight, relative to the total weight of the composition.

30. The composition according to claim 29, wherein the at least one diffracting pigment is present in an amount ranging from 0.5% to 5% by weight, relative to the total weight of the composition.

31. The composition according to claim 30, wherein the at least one diffracting pigment is present in an amount ranging from 0.5% to 2.5% by weight, relative to the total weight of the composition.

32. The composition according to claim 31, wherein the at least one diffracting pigment is present in an amount equal to 1% by weight, relative to the total weight of the composition.

33. The composition according to claim 4, wherein the at least one goniochromatic coloring agent is present in an amount ranging from 0.5% to 60% by weight, relative to the total weight of the composition.

34. The composition according to claim 4, wherein the at least one goniochromatic coloring agent is present in an amount less than or equal to 5% weight, relative to the total weight of the composition.

35. The composition according to claim 33, wherein the at least one goniochromatic coloring agent is present in an amount ranging from 0.5% to 2.5% by weight, relative to the total weight of the composition.

36. The composition according to claim 35, wherein the at least one goniochromatic coloring agent is present in an amount equal to 1% by weight, relative to the total weight of the composition.

37. The composition according to claim 1, further comprising at least one diffracting pigment comprising at least one layer of at least one reflecting material covered on at least one side by at least one layer of at least one-dielectric material.

38. The composition according to claim 37, wherein the at least one reflecting material is chosen from: Al; Ag; Cu; Au; Pt; Sn; Ti; Pd; Ni; Co; Rd; Nb; Cr; and alloys thereof;.and wherein the at least one dielectric material is chosen from: MgF₂; SiO₂; Al₂O₃; AlF₃; CeF₃; LaF₃; NdF₃; SmF₂; BaF₂; CaF₂; and LiF.

39. The composition according to claim 1, comprising at least one diffracting pigment comprising at least one layer of at least one dielectric material covered by at least one layer of at least one reflecting material on at least one side.

40. The composition according to claim 39, wherein the at least one dielectric material is chosen from: MgF₂; SiO; SiO₂; Al₂O₃; TiO₂; WO; AlN; BN; B₄C; WC; TiC; TiN; N₄Si₃; ZnS; particles of glass; and particles of diamond type carbons.

41. The composition according to claim 1, comprising at least one goniochromatic coloring agent comprising at least one material chosen from: MgF₂; CeF₃; ZnS; ZnSe; Si; SiO₂; Ge; Te; Fe₂O₃; Pt; Va; Al₂O₃; MgO; Y₂O₃; S₂O₃; SiO; HfO₂; ZrO₂; CeO₂; Nb₂O₃; Ta₂O₅; TiO₂; Ag; Al; Au; Cu; Rb; Ti; Ta; W; Zn; MoS₂; cryolite; alloys, and polymers.

42. The composition according to claim 4, wherein the composition does not comprise at least one goniochromatic coloring agent comprising at least one layer of reflecting metal.

43. The composition according to claim 42, wherein the reflecting metal is aluminum.

44. The composition according to claim 4, comprising at least one additional non-goniochromatic coloring agent.

45. The composition according to claim 44, wherein the at least one additional non-goniochromatic coloring agent is chosen from dyes and monochromatic pigments.

46. The composition according to claim 1, wherein the composition is provided in a form chosen from: anhydrous forms; oily and aqueous solutions; oily and aqueous gels; oil-in-water and water-in-oil emulsions; multiple emulsions; and dispersions of oil in water by virtue of vesicles situated at the oil/water interface.

47. A nail varnish comprising at least one composition having, for an incidence of illumation of 45°, a variation Dₕ of hue angle h of at least 50°, when the angle of observation is varied from −10° to +30°, and a difference (Δa*²+Δb*2)^{1/2} of at least 5 when the angle of observation is varied from −70° to −30°.

48. A makeup for the lips comprising at least one composition having, for an incidence of illumation of 45°, a variation Dₕ of hue angle h of at least 50°, when the angle of observation is varied from −10° to +30°, and a difference (Δa*²+Δb*2)^{1/2} of at least 5 when the angle of observation is varied from −70° to −30°.

49. A mascara comprising at least one composition having, for an incidence of illumation of 45°, a variation Dₕ of hue angle h of at least 50°, when the angle of observation is varied from −10° to +30°, and a difference (Δa*²+Δb*²)^{1/2} of at least 5 when the angle of observation is varied from −70° to −30°.

50. A foundation makeup comprising at least one composition having, for an incidence of illumation of 45°, a variation Dₕ of hue angle h of at least 50°, when the angle of observation is varied from −10° to +30°, and a difference (Δa*²+Δb*2)^{1/2} of at least 5 when the angle of observation is varied from −70° to −30°.

51. A method for making up at least one keratin material chosen from skin, lips, nails, and hair comprising applying at least one non-opaque goniochromatic coloring agent and at least one diffracting pigment.

52. The method according to claim 51, comprising applying the at least one non-opaque goniochromatic coloring agent and at least one diffracting pigment simultaneously.

53. A method for making up at least one keratin material chosen from skin, lips, nails, and hair comprising applying at least one composition comprising, in a physiologically acceptable medium, at least one non-opaque goniochromatic coloring agent and at least one diffracting pigment.

54. A composition, comprising, In a physiologically acceptable medium, from 0.5% to 2.5% by weight, relative to the total weight of the composition, of at least one diffracting pigment having a structure MgF₂/Al/MgF₂ and from 0.5% to 2.5% by weight, relative to the total weight of the composition, of at least one goniochromatic coloring agent having a structure Fe₂O₃/SiO₂/Fe₂O₃/SiO₂/Fe₂O₃, wherein the weight ratio of the at least one diffracting pigment to the at least one goniochromatic coloring agent ranges from 15/85 to 85/15.

55. The composition according to claim 54, wherein the at least one diffracting pigment is present in an amount equal to 1% by weight, relative to the total weight of the composition.

56. The composition according to claim 54, wherein the at least one goniochromatic coloring agent is present in an amount equal to 1% by weight, relative to the total weight of the composition.

57. The composition according to claim 54, wherein the weight ratio of the at least one diffracting pigment to the at least one goniochromatic coloring agent is equal to 50/50.

58. A composition, comprising, in a physiologically acceptable medium, from 0.5% to 2.5% by weight, relative to the total weight of the composition, of at least one diffracting pigment having a structure MgF₂/Al/MgF₂ and from 0.5% to 2.5% by weight, relative to the total weight of the composition, of at least one goniochromatic coloring agent having a structure Fe₂O₃/SiO₂/mica-oxide/SiO₂/Fe₂O₃, wherein the weight ratio of the at least one diffracting pigment to the at least one goniochromatic coloring agent ranges from 15/85 to 85/15.

59. The composition according to claim 58, wherein the at least one diffracting pigment is present in an amount equal to 1% by weight, relative to the total weight of the composition.

60. The composition according to claim 58, wherein the at least one goniochromatic-coloring agent is present in an amount equal to 1% by weight, relative to the total weight of the composition.

61. The composition according to claim 58, wherein the weight ratio of the at least one diffracting pigment to the at least one goniochromatic coloring agent is equal to 50/50.

62. A composition, comprising, in a physiologically acceptable medium, from 0.5% to 2.5% by weight, relative to the total weight of the composition, of at least one diffracting pigment having a structure MgF₂/Al/MgF₂ and from 0.5% to 2.5% by weight, relative to the total weight of the composition, of at least one goniochromatic coloring agent having a structure TiO₂/SiO₂/TiO₂, wherein the weight ratio of the at least one diffracting pigment to the at least one goniochromatic coloring agent ranges from 15/85 to 85/15.

63. The composition according to claim 62, wherein the at least one diffracting pigment is present in an amount equal to 1% by weight, relative to the total weight of the composition.

64. The composition according to claim 62, wherein the at least one goniochromatic coloring agent is present in an amount equal to 1% by weight, relative to the total weight of the composition.

65. The composition according to claim 62, wherein the weight ratio of the at least one diffracting pigment to the at least one goniochromatic coloring agent is equal to 50/50.

66. A composition, comprising, in a physiologically acceptable medium, from 0.5% to 2.5% by weight, relative to the total weight of the composition, of at least one diffracting pigment having a structure MgF₂/Al/MgF₂ and from 0.5% to 2.5% by weight, relative to the total weight of the composition, of at least one goniochromatic coloring agent having a structure TiO₂/Al₂O₃/TiO₂, wherein the weight ratio of the at least one diffracting pigment to the at least one goniochromatic coloring agent ranges from 15/85 to 85/15.

67. The composition according to claim 66, wherein the at least one diffracting pigment is present in an amount equal to 1% by weight, relative to the total weight of the composition.

68. The composition according to claim 66, wherein the at least one goniochromatic coloring agent is present in an amount equal to 1% by weight, relative to the total weight of the composition.

69. The composition according to claim 66, wherein the weight ratio of the at least one diffracting pigment to the at least one goniochromatic coloring agent is equal to 50/50.
